(19) United States
(12) Patent Application Publication
(10) Pub. No.: US 2025/0041122 A1
(43) Pub. Date: Feb. 6, 2025

(54) ARTICULABLE OTOLOGIC SUCTION TUBE AND SUCTION TUBE SYSTEM

(71) Applicant: Alcon Inc., Fribourg (CH)

(72) Inventors: Venjamin Stojanov, Dietlikon (CH); Wesley Hastings Elden, Southlake, TX (US)

(21) Appl. No.: 18/781,109

(22) Filed: Jul. 23, 2024

Related U.S. Application Data
(60) Provisional application No. 63/517,387, filed on Aug. 3, 2023.

(51) Int. Cl. A61F 11/20; A61M 25/01

(52) U.S. Cl. CPC A61F 11/202; A61M 25/0138

(57) ABSTRACT

An otologic instrument includes a handle configured to be held by a hand of a surgeon and a cannula extending outwardly from the handle and mounted to the handle. An articulated tube is at least partially positioned within the cannula and at least partially extends outwardly from a distal end of the cannula. An actuator is configured to articulate the articulated tube relative to the cannula to position a distal end of the suction tube at a plurality of positions within a three-dimensional volume. The actuator may be configured to control extension of the articulated tube relative to the cannula, the articulated tube having a curved shape when undeformed. The actuator may be configured to pull a cable or push a push rod within the articulated tube to adjust the curvature of the articulated tube.

### BACKGROUND

During middle ear surgery in otology, it is commonly necessary to suction various fluids, debris, and tissues, including infusion fluids, blood, tissues, and bone dust from the middle ear space. Suction tubes may be used for this purpose. Additionally, suction tubes may also be used for removal of cholesteatoma and infected tissues. Suction tubes can be particularly useful for reaching otherwise hard-to-reach cholesteatoma behind corners deep inside the ear.

At present, whenever a curved or a straight suction tube is needed during an otologic surgical procedure, a surgeon must change or swap the instrument(s) they are holding to perform the procedure. Furthermore, the possible curvature and reach of current suction tubes is often limited by the diameter of the patient's ear canal, since the curved suction tube is typically introduced through the ear canal.

These limitations can make it difficult for surgeons to apply minimally invasive surgical techniques during otologic procedures, since access for suction to the middle ear space typically requires that the suction tube be inserted into the ear canal and through an incision adjacent the tympanic membrane into the middle ear space.

### SUMMARY

In certain embodiments, an otologic instrument includes a handle configured to be held by a hand of a surgeon and a cannula extending outwardly from the handle and mounted to the handle. An articulated tube is at least partially positioned within the cannula and at least partially extends outwardly from a distal end of the cannula. An actuator is configured to articulate the articulated tube relative to the cannula to position a distal end of the articulated tube at a plurality of positions within a three-dimensional volume.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a schematic cross-sectional view of a human ear of the prior art.

FIG. 2 is an isometric view of an otologic instrument in accordance with certain embodiments.

FIGS. 3A and 3B are side views of a first embodiment of an articulated tube in accordance with certain embodiments.

FIG. 4A is a side view of a second embodiment of an articulated tube in accordance with certain embodiments.

FIGS. 4B to 4D are cross sectional views of the second embodiment of the articulated tube.

FIGS. 5A and 5B are side views illustrating alternative embodiments for an otologic instrument in accordance with certain embodiments.

FIG. 6A is a side view of a first embodiment of an instrument housing in accordance with certain embodiments.

FIG. 6B is a cross sectional view of the first embodiment of the instrument housing along section line 6B.

FIG. 6C is a front view of the first embodiment of the instrument housing.

FIG. 7A is a side view of a second embodiment of an instrument housing in accordance with certain embodiments.

FIG. 7B is a cross sectional view of the first embodiment of the instrument housing along section line 7B.

FIG. 7C is a cross sectional view of the first embodiment of the instrument housing along section line 7C.

FIGS. 8A to 8K illustrate various mechanisms for increasing tension of a pull-wire in surgical instruments in accordance with certain embodiments.

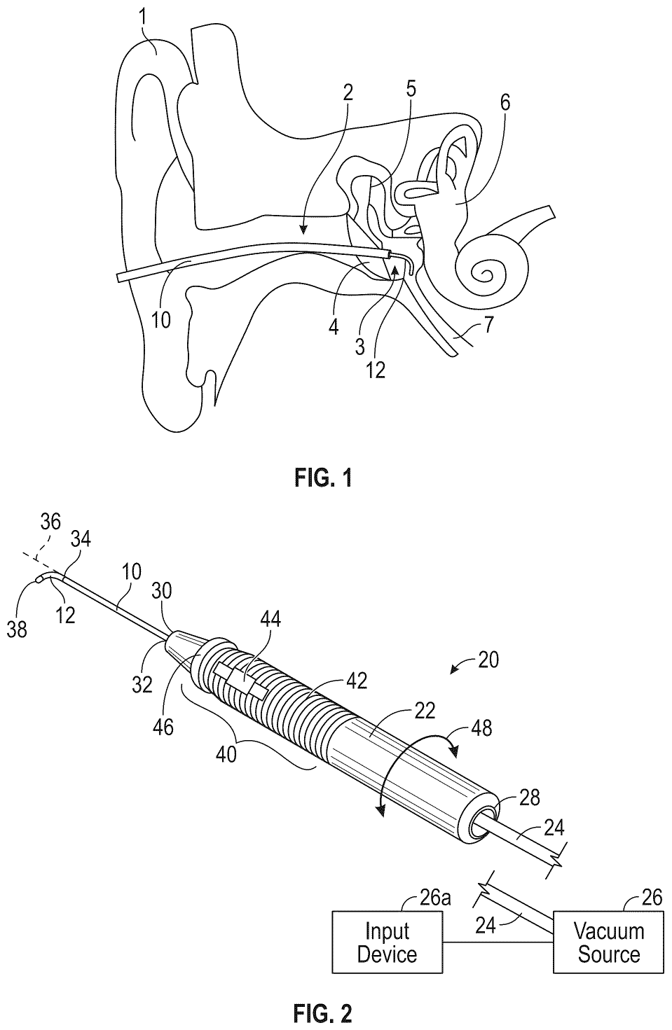

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The human ear includes the pinna 1, which is the fleshy part of the ear protruding outwardly from the skull and the ear canal 2, which passes through the skull to the middle ear 3. The adult human ear canal 2 extends from the pinna 1 to the tympanum 4 and is about 2.5 cm (centimeters) in length and 0.7 cm in diameter. The tympanum 4, or eardrum, separates the ear canal 2 from the middle ear 3. The tympanum 4 is coupled by ossicles 5 to the cochlea, which includes vibration sensitive hairs for detecting sound. The middle ear 3 is further coupled to the sinuses of the skull by the eustachian tube 7.

For trans-ear canal procedures, a cannula 10 is inserted through the ear canal 2 into the middle ear 3. The cannula 10 is typically inserted through a tympanomeatal flap. The tympanomeatal flap is created by making a series of incisions in the tissue around the tympanum 4. The flap is then elevated to gain access to the middle ear 3 behind the tympanum 4, such as by inserting the cannula 10. Surgical instruments may be passed through the lumen of the cannula 10. In a typical otologic procedure, suction will be applied to the middle ear 3 to remove infected tissue, pus, cholesteatoma, infusion fluids, blood, or debris resulting from the otologic procedure, such as bone fragments and other tissue. Accordingly, an articulated tube 12 may pass through the cannula 10 into the middle ear 3 to suction material from the middle ear 3. In some instances, the articulated tube 12 may also be used to grasp tissue or as an additional instrument for manipulating tissue by pushing.

Many different conditions affect the middle ear space with resulting hearing loss for the affected patients. Often the hearing loss needs to be treated by middle ear surgery that requires inserting precision tools into the ear, such as through the ear canal 2. Diagnosis and surgical treatment present significant challenges due to difficult visualization, limited accessibility, and the delicate nature of the concerned anatomical structures (e.g., ossicles 5, tympanum 4, oval window, etc.). These difficulties and limitations make it difficult for surgeons to apply minimally invasive techniques and they must often utilize more invasive approaches (e.g., postauricular approach or mastoidectomy) to realize sufficiently good visualization and accessibility to treat the patient's condition successfully.

Accordingly, to reach certain areas, the articulated tube 12 may need to be curved. The degree of curvature and the orientation of the plane of curvature needed will vary throughout the suctioning process. The articulated tube 12 may be implemented as an articulated tube 12 according to any of the embodiments disclosed herein to achieve the varying radiuses of curvature and orientations. In the following description, reference is made to trans-ear canal otologic procedures. It shall be understood that the embodiments disclosed herein provide at least some benefit when used in trans-mastoid procedures. For example, the articulated tube may be used when performing mastoidectomy to remove large cholesteatomas. In some instances, the same procedure may include both trans-mastoid and trans-canal access each using an articulated tube 12. The surgeon may use whichever of trans-mastoid and trans-canal access is better suited to access a particular piece of cholesteatoma.

Referring to FIG. 2, a surgical instrument 20 includes a handle 22 that is held in the hand of a surgeon performing an otologic procedure. In a typical procedure, the other hand of the surgeon will manipulate a second instrument while using the surgical instrument 20 to provide suction. However, as discussed below, the surgical instrument 20 may implement functions in addition to providing suction.

The instrument 20 is coupled by tube 24 to a vacuum source 26, such as a vacuum pump. The supply of vacuum pressure through the tube 24 may be controlled by an input device 26a, such as a foot pedal operated by the surgeon, a button provided on the handle 22, or some other input device, such as a touch screen, voice command processing device, gesture detection device, or the like. In the illustrated embodiment, the tube 24 enters the handle 22 through a proximal end 28 of the handle 22 with the cannula 10 protruding from the distal end 30 of the handle 22.

The cannula 10 extends from a proximal end 32 secured to the handle 22 to a distal end 34 of the cannula 10. The cannula 10 may be straight between the proximal end 32 and the distal end 34 or may be curved. For example, the cannula 10 may be curved to conform generally to the curvature of the ear canal 2. The cannula 10 may be implemented as a tube made of metal such as nitinol, steel, or aluminum or a rigid or flexible polymer. The cannula 10 may be configured to remain substantially undeformed during use or may flex to conform to the ear canal 2 or responsive to movements of the handle when in place in the ear canal 2 with the distal end 34 of the cannula 10 within the middle ear 3.

The articulated tube 12 protrudes from the distal end 34 of the cannula 10 and extends to a distal end 38 of the articulated tube 12. The distal end 38 defines an opening for suctioning material. The opening may be at the end face of the articulated tube 12 or the end of the articulated tube 12 may be closed and one or more openings may be formed on the side of the distal portion of the articulated tube 12 for suctioning material. The articulated tube 12 will flex during operation and therefore is made of a material that may be elastically flexed without failing. The ability of the articulated tube 12 to bend may be due to properties of the material and/or features formed in the articulated tube 12 as discussed below. The articulated tube 12 may therefore be made of metal such as nitinol, steel, or aluminum or a rigid or flexible polymer. The articulated tube 12 is sized to fit within a lumen of the cannula 10, which is itself sized to fit within the ear canal 2.

In various embodiments, the cannula 10 has a total length that ranges from 4-15 cm and an outer diameter that ranges from 0.4-10.0 mm (millimeters), more particularly 0.4.0-5.0 mm, and even more particularly 0.4-3.0 mm. The articulated tube 20 may have a length that ranges from 0.5-2.0 cm and an outer diameter that ranges from 0.4.0-10.0 mm, more particularly 0.4-5.0 mm, and even more particularly 0.4-3.0 mm.

The portion of the articulated tube 12 between the distal end 34 of the cannula 10 and the distal end 38 of the articulated tube 12 is able to assume a plurality of radiuses of curvature in a plurality of bending planes, such as bending planes substantially, e.g., within 0.5 mm, intersecting an axis 36 of the cannula 10, the axis 36 being parallel to and collinear with the axis of symmetry of the lumen of the cannula 10. Where the cannula 10 is curved, the axis 36 may be defined as a line extending from the distal end 34 of the cannula and tangent to a curve defined by the center line of the cannula 10 at a point on the curve within 0.1 mm from the distal end 34. The length of the suction tube 12 between the distal end 34 of the cannula 10 and the distal end 38 of the suction tube 12 may also be adjustable.

For example, a section 40 of the handle 22 may include a grip 42 for engaging the fingers of the surgeon holding the handle 22. One or more control structures may be positioned on or near the section 40. For example, the one or more control structures may include one or more sliders 44 mounted to the handle 22 and directly or indirectly coupled to the cannula 10 or the suction tube 12. The slider 44 may be engaged by the thumb or other finger of the surgeon to adjust the relative positions of the cannula 10 and the suction tube 12. For example, movement of the slider 44 toward the distal end 30 of the handle 22 may extend the cannula 10 over the suction tube 12 thereby retracting the suction tube 12 relative to the cannula 10. Movement of the slider 44 toward the proximal end 28 of the handle 22 may retract the cannula 10 into the handle 22 thereby extending the suction tube 12 relative to the cannula 10. Where the slider 44 is coupled to the suction tube 12, the movements are reversed: movement of the slider 44 toward the distal end 30 will extend the suction tube 12 relative to the cannula 10 and movement of the slider 44 toward the proximal end 28 will retract the articulated tube 12 relative to the cannula 10. Although a slider 44 is shown, other actuators may be used, such a deformable basket, depressible button, or other manually actuated structure.

One or both of the cannula 10 and the articulated tube 12 may be rotatable relative to the handle 22. The articulated tube 12 may therefore be coupled to the tube 24 by a rotary pneumatic junction to facilitate such rotation while still maintaining a seal between the articulated tube 12 and the tube 24. For example, the control structures mounted to the handle 22 may include a dial 46 or other control structure such that actuation of the dial 46 will rotate one or both of the cannula 10 and the articulated tube 12 relative to the handle 22. In this manner, the plane of curvature of the articulated tube 12 may be within a variety of planes intersecting the axis 36. Stated differently, the distal end 38 of the articulated tube 12 may be positioned at a plurality of points within a three-dimensional volume around the distal end 34 of the cannula 10 by adjusting the radius of curvature of the articulated tube 12 and the orientation of the articulated tube 12 about the axis 36.

Note that in some embodiments, the cannula 10 and the articulated tube 12 are not rotatable relative to the handle 22. In such embodiments, the surgeon may perform rotational movement 48 of the handle 22 itself to achieve the same range of movement for the distal end 38 of the articulated tube 12.

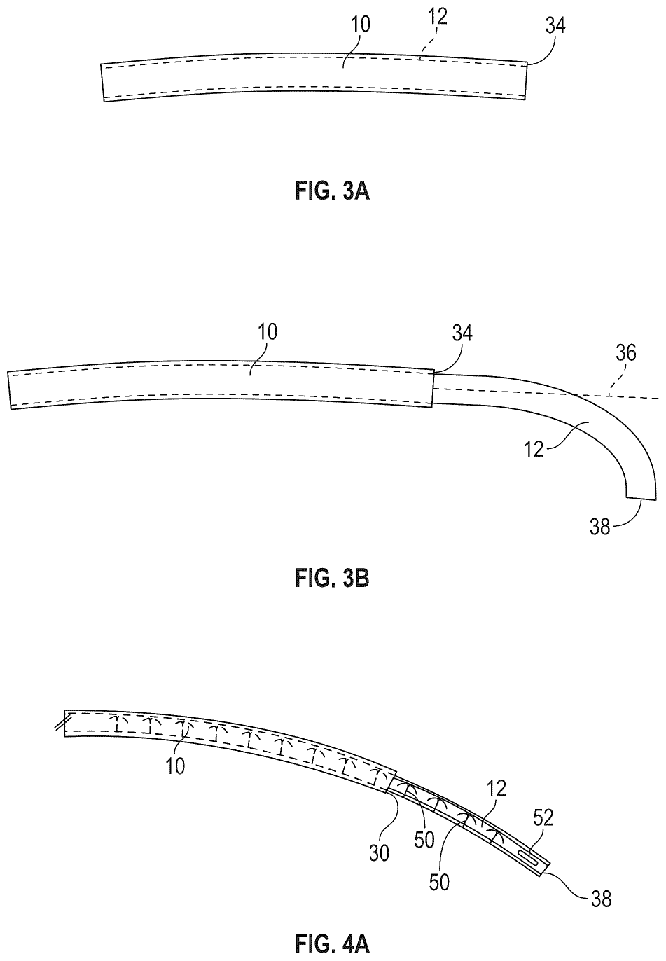

Referring to FIGS. 3A and 3B, in some embodiments, articulation of the articulated tube 12 is performed in coordination with the cannula 10. In the illustrated embodiment, the suction tube 12 in an undeformed state has the curved shape shown in FIG. 3B. The suction tube 12 must therefore deform to fit within the cannula 10. However, positioning within the cannula 10 requires only elastic deformation of the suction tube 12. The radius of the undeformed articulated tube 12 may be constant or may vary with distance from the distal end 38. For example, in the illustrated embodiment, the radius of curvature decreases with distance from the distal end 38. In other embodiments, the radius of curvature may increase with distance from the distal end 38. The radius of curvature of the distal end 38 may have a single value or include a range of values between 0.1 mm and 10 mm.

The distal end 38 of the articulated tube 12 may be placed at various positions relative to the distal end 34 of the cannula 10 by changing the length of the articulated tube 12 extending outwardly from the distal end 34 of the cannula 10. The further the articulated tube 12 is extended, the greater the distal end 38 will extend radially outwardly from the axis 36 of the cannula 10. This range of movement of the articulated tube 12 coupled with a range of translational movement of the cannula 10 itself enables the distal end 38 of the articulated tube 12 to be arbitrarily positioned in three dimensions within the middle ear 3, including extending around obstructions.

In some embodiments, the articulated tube 12 is made of a formable material such that a surgeon may deform the articulated tube into a desired shape either manually or using a tool. The articulated tube 12 may then be extended out of the cannula 10 to a desired point within the middle ear 3, which may include rotation of the articulated tube 12 as described above. The deformation required to pass the articulated tube 12 through the cannula 10 may be less than required to completely or partially undo the deformation performed by the surgeon. Using this approach, the surgeon may form the articulated tube 12 into multiple shapes throughout an otologic procedure to access desired locations within the middle ear 3.

As yet another alternative, a surgeon may be provided with an array of articulated suction tubes 12 with different radiuses of curvature, different curve shapes, different lengths, or other variations in properties and may mount a selected articulated suction 12 to the handle 22 at any point during a procedure to gain access to desired locations within the middle ear 3.

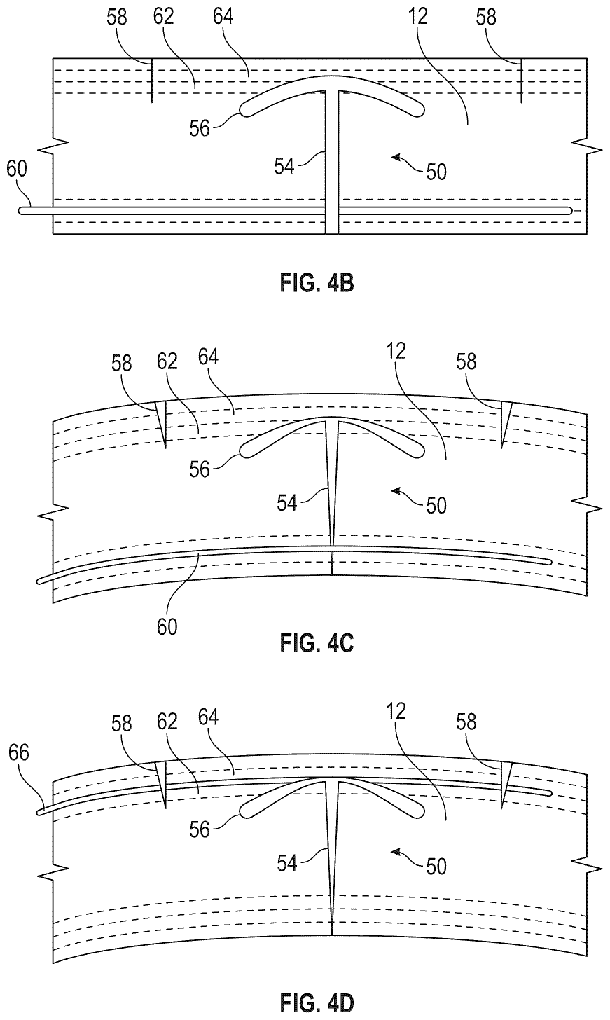

Referring to FIGS. 4A, 4B, and 4C, in an alternative embodiment, the articulated tube 12 may be actuated by means of a cable, push rod or other actuator to change the radius of curvature of the articulated tube 12 and the corresponding position of the distal end 38. In such embodiments, the undeformed shape of the articulated tube 12 may be straight or curved with a constant or varying radius of curvature.

The articulated tube 12 may include one or more regions having different mechanical properties relative to the remainder of the articulated tube 12 to facilitate bending of the articulated tube 12, such as bending in a primary bending plane. In the illustrated embodiment, the primary bending plane is parallel to the page. The one or more regions of differing mechanical properties along the length of the articulated tube 12 are such that the force required to bend the articulated tube 12 in the primary bending plane is less than 50 percent, less than 25 percent, or less than 10 percent of the force required to bend the articulated tube 12 in a plane defined as being perpendicular to the primary bending plane and intersecting the center line of the articulated tube 12 at one, two, or more points, such as when the articulated tube 12 is straight. The one or more regions of differing mechanical properties along the length of the articulated tube 12 may also be such that the force required to bend the articulated tube 12 in one direction in the primary bending plane (“the primary direction”) is less than 50 percent, less than 25 percent, or less than 10 percent of the force required to bend the articulated tube 12 in the opposite direction in the primary bending plane.

In the illustrated embodiment, each region of the one or more regions of differing mechanical properties is implemented as a notch 50 extending partially through the articulated tube 12. The notches 50 may be cut by means of laser cutting, co-molding with the articulated tube 12, or other cutting process. The notches 50 may be cut through the articulated tube 12 perpendicular to the primary bending plane. The notches 50 may extend inwardly into the articulated tube 12 from the concave side of the articulated tube 12 when bent in the primary direction in the primary bending plane (hereinafter “the concave side”), from the side opposite the concave side (hereinafter “the convex side”), or both. The notches 50 may be distributed along all or part (e.g., at least 50 percent) of a portion of the articulated tube 12 that extends outwardly from the cannula 10 when the articulated tube 12 at the maximum extension of the articulated tube 12.

In the illustrated embodiment, a distal portion, e.g., the distal 1 to 10 mm, of the articulated tube 12 includes one or more lateral slots 52 passing there through. The distal portion may lack notches 50. The one or more lateral slots 52 may be cut through the articulated tube 12 perpendicular to the bending plane. In the illustrated embodiment, the one or more lateral slots 52 have the long dimension thereof oriented substantially (e.g., within 5 degrees of) parallel to the center line of the articulated tube 12 in which the one or more lateral slots 52 are formed. The slots 52 may facilitate bending and deformation of the distal portion whether in response to an actuation force or external forces acting on the distal portion.

Referring specifically to FIGS. 4B and 4C, each slot 50 may include a portion 54 that extends through the articulated tube 12 from one side of the articulated tube 12, such as extending inwardly from the concave side. The portion 54 has a width parallel to the center line of the articulated tube 12 that is diminished when the articulated tube 12 is bent in the primary direction in the primary bending plane as shown in FIG. 4C. When the articulated tube 12 is undeformed, the width may be between 0.01 and 0.1 times the outer diameter of the articulated tube 12.

Each slot 50 may include an arcuate portion 56 extending to either side of the portion 54, such as across the deepest point of the portion 54. The arcuate portion 56 may are downwardly toward the center line of the articulated tube 12 and/or toward the concave side. The arcuate portion 56 may be symmetric or asymmetric with respect to the center of the portion 54. When the articulated suction tube is undeformed, the arcuate portion 56 may have a radius of curvature between 0.5 and 1 times the outer diameter of the articulated tube 12 and may have an extent along the center line of the articulated tube 12 when straight of between 0.3 and 1 times the outer diameter of the articulated tube 12. When the articulated tube 12 is undeformed, the thickness of the arcuate portion 56 may be between 0.01 and 0.1 times the outer diameter of the articulated tube 12. As shown in FIG. 4C, the thickness of the arcuate slot 56 is diminished when the articulated tube 12 is bent in the primary direction in the primary bending plane.

In some embodiments, the convex side may have expansion slots 58 extending inwardly therefrom. The depth of the expansion slots 58 may be less than the depth of the slots 50 inward from the concave side, such as less than 50 percent, less than 25 percent, or less than 10 percent. When undeformed, the expansion slots 58 may have a small thickness parallel the center line of the articulated tube 12 when straight, such as no wider than the kerf width of the process used to form the expansion slots 58. For example, the expansion slots 58 may have a thickness that is less than 0.1 mm, 0.01 mm, or less than one micrometer. In the illustrated embodiments, the expansion slots 58 are positioned between slots 50, such as at a midpoint between slots 50. As shown in FIG. 4C, the expansion slots 58 are widened when the articulated tube 12 is bent in the primary direction in the primary bending plane.

The configuration of the slots 50, 52, 58 is exemplary only. Other configurations may be used. For example, the articulated tube 12 may be implemented using any of the slotted tip designs of FIGS. 3A to 3H of U.S. Pat. No. 10,085,883, which is hereby incorporated herein by reference in its entirety.

A cable 60 extends within the articulated tube 12 such that the cable 60 extends across a plurality of the slots 50 and secures to the articulated tube 12 near, e.g., within 10 mm, 5 mm, 1 mm, or 0.1 mm of the distal end 38 of the articulated tube 12. In the illustrated embodiment, the cable 60 extends within the articulated tube 12 and is secured to the articulated tube 12 closer to the concave side than the convex side such that the cable 60 may be tensioned to bend the articulated tube 12 in the primary direction in the primary bending plane. The cable 60 may secure to the suction tube 12 within the distal portion as defined above or at some other point offset a greater extent from the distal end 38.

Referring to FIG. 4D, in other embodiments, the cable 60 may be implemented as a push rod 66 that is pushed. The push rod may extend along and be secured to the articulated tube 12 closer to the convex side than to the concave side.

The articulated tube 12 may be made of an elastic material such that when tension is removed on the cable 60 or pressure on a push rod 66 is removed, the articulated tube 12 will recoil to an undeformed shape to the extent permitted by any resistance of material within the middle ear 3 contacting the suction tube 12.

In embodiments including a cable 60 or push rod 66, the slider 44 may be coupled to the cable 60 or push rod 66 to bend or unbend the articulated suction tube responsive to inputs from the surgeon. The slider 44 may be coupled to the cable 60 or a push rod 66 by means of any of the mechanisms shown in FIGS. 4A to 4D of U.S. Pat. No. 10,085,883. Alternatively, the cable 60 or push rod may be driven by an electrical, pneumatic, hydraulic, or other type of actuator subject to an input from the surgeon.

In some embodiments, the articulated tube 12 is actuated by the cable 60 or a push rod 66 and can also be extended and retracted relative to the cannula 10. Accordingly, a second slider 44 or other input device may be mounted to the handle 22 and coupled to the articulated tube 12 or cannula 10 to induce relative movement between the articulated tube 12 and the cannula 10 as described above with respect to the slider 44.

In the illustrated embodiment, one or more sleeves 62, 64 are positioned within the lumen of the articulated tube 12. At least one sleeve 62 may be used to provide a sealed path through the articulated tube 12 and prevent fluid flow through the slots 50, 52, 58 into the lumen of the sleeve 62. In some embodiments, an additional sleeve 64 may be positioned between the sleeve 62 and the articulated tube 12 with the cable 60 or push rod 66 positioned between the sleeves 62, 64 to retain the cable 60 or push rod 66 and isolate the cable 60 or push rod 66 from the environment of the articulated tube 12. The one or more sleeves 62, 64 may be made of the same material as the articulated tube 12 or different material. For example, the sleeves 62, 64 may be made of a polymer that is more flexible than that used to form the articulated tube 12, e.g., having a hardness that is at least 10, 25, or 50 lower on the Shore A scale or Shore D scale.

In the embodiments of FIGS. 3A, 3B, 4A, 4B, 4C, and 4D, movement of the articulated tube 12 in a single primary bending plane with ending in the primary direction (and corresponding recoiling movement) are discussed. However, the articulated tube 12 may be articulated in other ways. For example, multiple cables 60 and/or push rods 66 configured as described above may be provided with various positions around the circumference of the articulated tube 12 to provide bending in multiple bending planes. Likewise, slots may define various bending planes, such as a first pattern of slots defining a first bending plane and a second pattern of slots defining a second bending plane and oriented at 90 degrees relative to the first pattern of slots around the circumference of the articulated tube 12. Likewise, within a single bending plane, one cable 60 and/or push rod 66 may be used for bending in one direction with another cable and/or push rod used for bending in the other direction within. In such embodiment, the one or more regions of modified mechanical properties may either be omitted or be selected to facilitate bending in more than one bending plane.

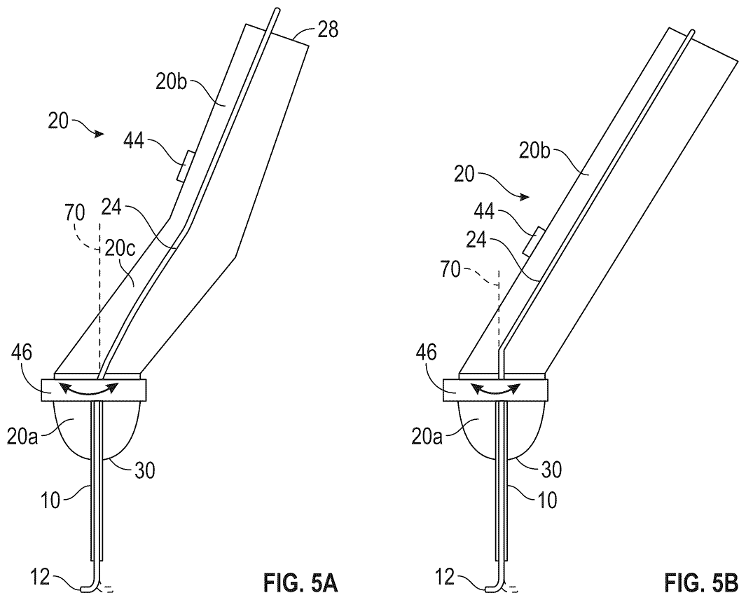

Referring to FIGS. 5A and 5B, the handle 20 may have various configurations. In the illustrated embodiments, the handle 20 includes a portion 20b that is one or both of offset and angled with respect to a portion 20a to which the cannula 10 is secured. The portion 20b is configured to be grasped by the hand of a surgeon and may have a longest dimension of between 10 and 20 centimeters. For example, the portion 20a may define an axis 70 that may be defined as an axis of symmetry of the portion 20a, an axis of rotation of the dial 46, or an axis of symmetry of the portion of the cannula 10 positioned within the portion 20a. The portion 20b may therefore be one or both of angled with respect to the axis 70 (FIGS. 5A and 5B) and offset radially from the axis 70. The angle of the portion 20b relative to the axis 70 may be defined as an angle between an axis of symmetry of the portion 20b, or the orientation of the longest dimension of a portion 20b that is asymmetric, and the axis 70. The offset may be achieved by a portion 20c joining the portion 20a to the portion 20b and extending outwardly from the axis 70. The offset and angle of the portion 20b relative to the portion 20a may ensure that the hand of the surgeon holding the portion 20b does not obstruct a view of the ear canal 3 during an otologic procedure. For example, the offset may be between 1 and 5 cm. The angle may be between 15 and 45 degrees, such as between 25 and 40 degrees with an overall length of the handle 20 between the proximal end 28 and the distal end 30 being between 15 and 25 centimeters. In the embodiments of FIGS. 5A and 5B, the slider 44 may be mounted to the portion 20a, 20b, or 20c. In particular, flexibility of the cable 60 may enable the cable 60 to accommodate the offset and/or change in angle for the embodiments of FIGS. 5A and 5B such that the slider 44 may be positioned at any ergonomic position on the portion 20a, 20b, or 20c.

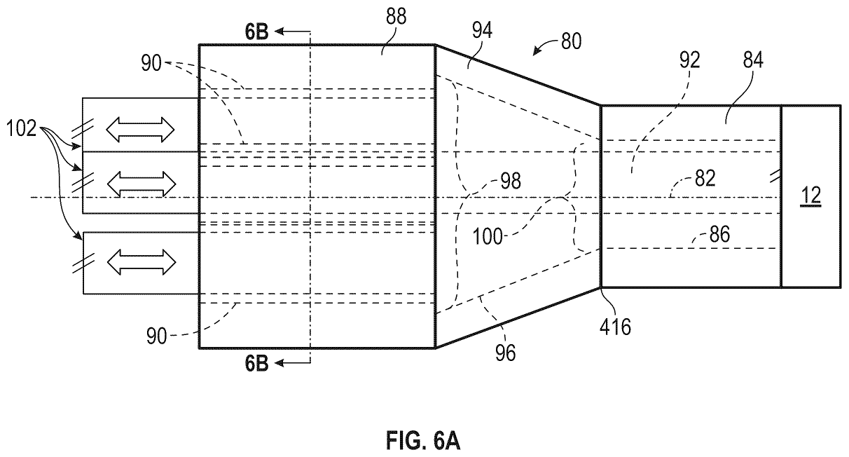

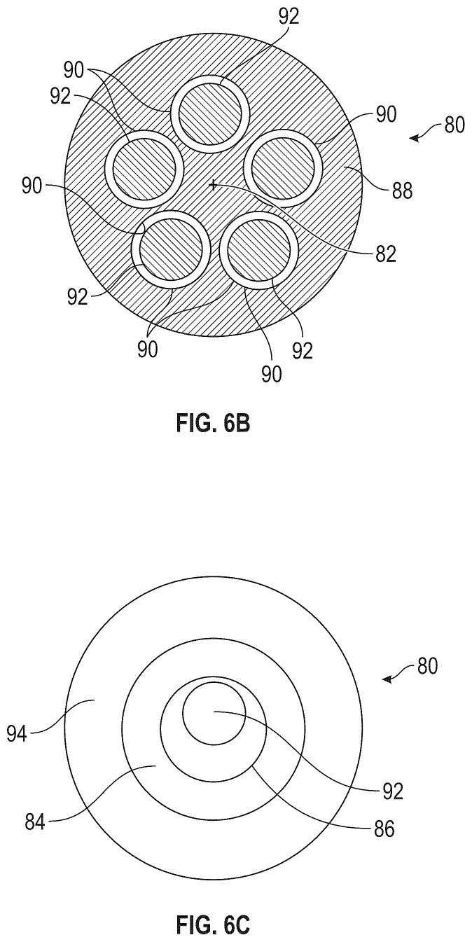

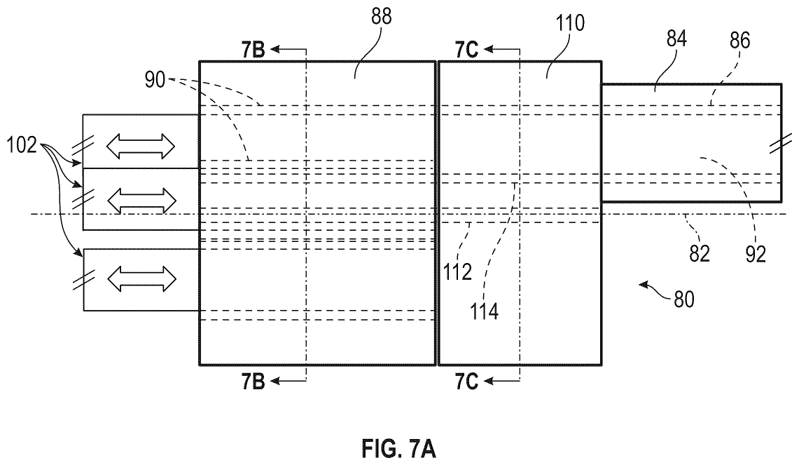

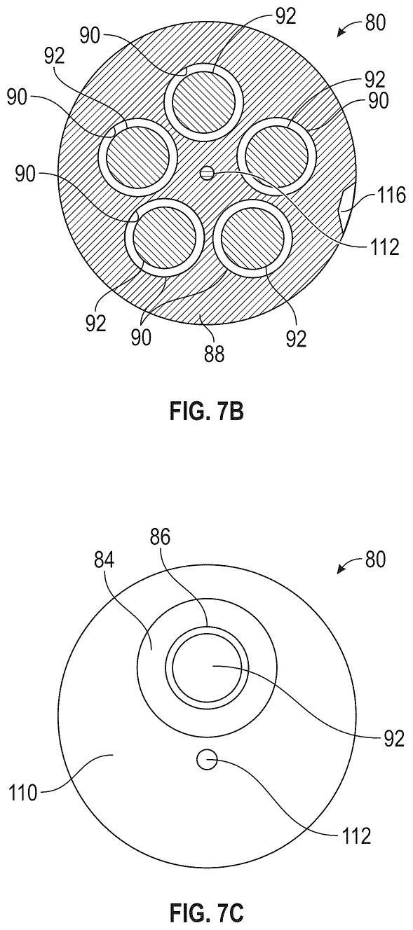

Referring to FIGS. 6A to 7C, various modifications of the instrument 20 described herein may be made. For example, the articulated tube 12 may serve an additional or alternative purpose. For example, a plurality of medical instruments that may be deployed using the articulated tube 12 may include some or all of the following:
A tube supplying vacuum pressure.
A tube for providing infusion fluid (e.g., saline).
A tube for infusing medication, gel, foam, viscoelastic material, or other material.
An optical fiber or fibers conducting light to the middle ear 3.
An optical fiber or fibers conducing light from the middle ear 3 to a camera, endoscope, or other imaging device.
An optical fiber or fibers for conducting laser light to the middle ear 3 for ablation or other treatments.
A cable coupled to an ultrasonic imaging transducer positioned at or near (e.g., within 2 mm) of the distal end 38 of the articulated tube 12.
A tube conducting pressurized gas for inflating or other purposes.
A waveguide conducting electromagnetic waves for performing diathermy.

Any of the above-listed medical instruments may be statically positioned within the articulated tube 12 either individually or as a group of two or more instruments.

Referring to FIGS. 6A to 7C, the instrument 20 according to any of the above-described embodiments may include an instrument housing 80 configured to deploy a plurality of medical instruments through the articulated tube 12 to provide a trans-canal medical assessment, diagnosis, treatment, or surgical procedure in the middle ear 3. Each medical instrument of the plurality of medical instruments may include either (a) an individual medical instrument of the medical instruments listed above or (b) two or more of the above-listed medical instruments combined into a single structure or otherwise deployed as single medical instrument as described below.

The instrument housing 80 enables each medical instrument of the plurality of medical instruments to be selectively deployed one at a time from a single staging location in any sequence and guided into the articulated tube 12. In the embodiments of FIGS. 6A to 7C, the plurality of medical instruments are housed in the instrument housing 80, which is disposed ex vivo in an external environment for use by the surgeon or other medical professional. The instrument housing 80 is operably coupled to the articulated tube 12, such as by attachment to a proximal end of the articulated tube 12. The instrument housing 80 may be mounted to the handle 22 or may be remote from the handle 22 and coupled to the articulated tube 12 by the tube 24 or some other tube.

Referring specifically to FIGS. 6A, the instrument housing 80 may define a longitudinal axis 82. The longitudinal axis 82 may be substantially (e.g., within 5 degrees of) parallel to and substantially (e.g., within 0.1 mm) collinear with the center line of the articulated tube 12 at a point of attachment of the tube 12 to the instrument housing 80. The components of the instrument housing 80 may be made of metal, such as stainless steel, aluminum, or nitinol or a rigid polymer.

The instrument housing 80 may include a portion 84 defining a lumen 86, such as a cylindrical lumen that is cylindrical about the longitudinal axis 82. The exterior of the portion 84 may likewise be cylindrical and centered on the longitudinal axis 82. The lumen 86 may have a diameter that is substantially (e.g., within 0.1 mm) identical to the diameter of the lumen of the articulated tube 12. The articulated tube 12 abuts the portion 84 to receive medical instruments inserted through the lumen 86.

The instrument housing 80 includes a receiver 88 defining a plurality of receptacles 90. The receiver 88 may have a cylindrical outer surface centered on the axis 82 or some other exterior shape. The receptacles 90 may be embodied as cylindrical bores having the axes thereof substantially (e.g., within 5 degrees of) parallel to the axis 82. The receptacles 90 may have any distribution within the receiver 88 and have any number that fits within the receiver 88 while being large enough to receive a medical instrument of the plurality of medical instruments. For example, there may be from 2 to 20 or from 2 to 5 receptacles 90. For example, referring to FIG. 6B, there may be five receptacles 90 distributed uniformly around the axis 82. The receptacles 90 extend completely through the receiver 90 parallel to the axis 82. Each receptacle 90 receives an instrument 92 of the plurality of medical instruments, which may have a cylindrical or non-cylindrical shape. The receptacles 90 are sized to permit the instruments 92 to freely slide through the receptacles 90.

The receiver 88 is coupled to the portion 84 by a tapered portion 94. The tapered portion 94 has a tapered lumen 96 that provides a smooth transition between the receiver 88 and the lumen 86. For example, the tapered lumen 96 may have a frusto-conical shape. The outer surface of the tapered portion 94 may likewise have a frusto-conical shape. A proximal opening 98 of the lumen 96 is sized to overlap the receptacles 90 and may be sized to extend outwardly perpendicular to the axis 82 from all of the receptacles 90, such as outwardly from 1 to 3 mm. A distal opening 100 of the tapered portion 94 may be substantially identical (e.g., within 0.1 mm) in size to the lumen 86 of the portion 84. An edge between the tapered lumen 96 and the lumen 84 may be beveled or chamfered to facilitate passage of a medical instrument into the lumen 84. The length of the tapered lumen 96 along the axis 82 and the corresponding cone angle of the lumen 96 may be selected to smoothly guide each medical instrument 92 from a receptacle 90 into the lumen 84. The length and cone angle may be selected based on flexibility of the instruments 92 being used. For example, the length may be between one and three times the diameter of the lumen 84 and the cone angle may be between 15 and 45 degrees.

Each receptacle may have a corresponding manipulation device 102 coupled thereto or substantially (e.g., within 0.1 mm) aligned therewith in a plane perpendicular to the axis 82. The manipulation device 102 may be embodied as a manually actuated actuator or an actuator controlled by means of electrical signals or hydraulic or pneumatic pressure. Each manipulating device engages an instrument 92 to translate the instrument 92 out of a corresponding receptacle 90, through the tapered lumen 96, through the lumen 86, and into the articulated tube 12. The manipulation device 102 is likewise configured to withdraw the instrument 92 into the receptacle 90 following use. The manipulation devices 102 may be manually actuated by a surgeon or activated by a robot or computer system coupled to the manipulation devices 102 in response to inputs received from the surgeon.

In use, the surgeon will select an instrument 92 for use and actuate or invoke actuation of the manipulation device 102 corresponding to the receptacle 90 housing the instrument 92. The manipulation device 102 will then urge the instrument 92 through the tapered lumen 96, lumen 84, and into the actuated tube 12 (see FIG. 6C). Note that where a first instrument 92 is already present in the actuated tube 12, the process of deploying a second instrument 92 includes withdrawing the first instrument into the receptacle 90 corresponding to the second instrument 92 using the manipulation device 102 corresponding to that receptacle 90.

Referring to FIGS. 7A to 7C, in some embodiments of the instrument housing 88, the receiver 88 is rotatable relative to the portion 84. In such embodiments, the portion 84 may be offset from the axis 82, such as having a center offset by a same amount by which centers of the receptacles 90 of the receiver 88 are offset from the axis 82. The portion 84 may be statically mounted to a mounting portion 110. The receiver 88 is rotatably mounted to the mounting portion, such as by means of a journal pin 112 with the axis of rotation of the receiver 88 being substantially (e.g., within 2 degrees of) parallel to the axis 82 and substantially collinear (e.g., within 0.1 mm) with the axis 82. The mounting portion 110 defines a lumen 114 substantially aligned with (e.g., within 0.1 mm) the lumen 86 and substantially identical in diameter (e.g., within 0.1 mm) to the lumen 86 of the portion 84. The mounting portion 110 and portion 84 may be monolithically formed. The lumen 114 may be substantially identical in diameter (e.g., within 0.1 mm) to the receptacles 90 or may be larger. The lumen 114 may for example taper between a diameter larger than or substantially identical to that of the receptacles to a diameter that is substantially identical to that of the lumen 86.

The receiver 88 may be manually rotatable with respect to the mounting portion 110. A detent or other structure may facilitate alignment of the receptacles 90 with respect to the lumen 114. A grip 116, knurling, rubberized surface, or other structure may be provided on the outer surface of the receiver 88 to facilitate rotation thereof by the surgeon. Alternatively, rotation of the receiver 88 may be performed by an electronic, mechanical, pneumatic, or hydraulic actuator responsive to inputs from the surgeon. For example, the positioning of the receiver 88 by the actuator may be controlled by a computer responsive to inputs received by the computer from the surgeon.

In the embodiment of FIGS. 7A to 7C, the manipulation devices 102 may function as described above. In particular, assuming a first instrument 92 is currently positioned within the articulated tube 12, the embodiments of FIGS. 7A to 7C may include activating the manipulation device 102 corresponding to the a first receptacle 90 to withdraw the first instrument 92 into a first receptacle 90, rotating the receiver 88 to align a second receptacle 90 with the lumen 114 of the mounting portion 110, activating the manipulating device corresponding to the second receptacle 90 to translate a second instrument 92 through the lumen 114, the lumen 86, and into the articulated tube 12. In an alternative approach, a single manipulation device 102 is used and engages only whichever of the instruments 92 is in a receptacle 90 currently aligned with the lumen 114.

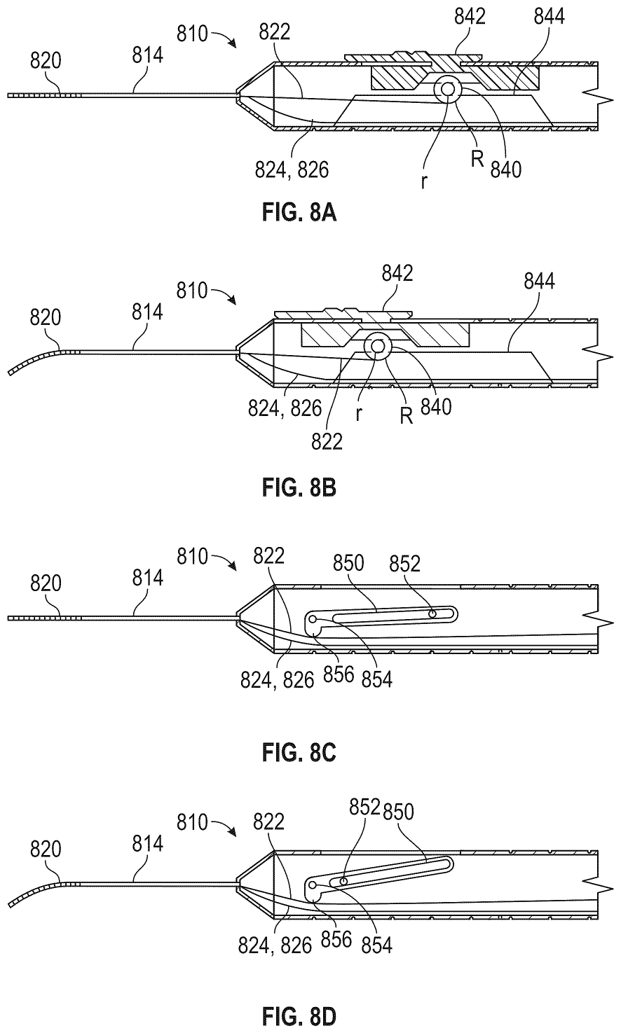

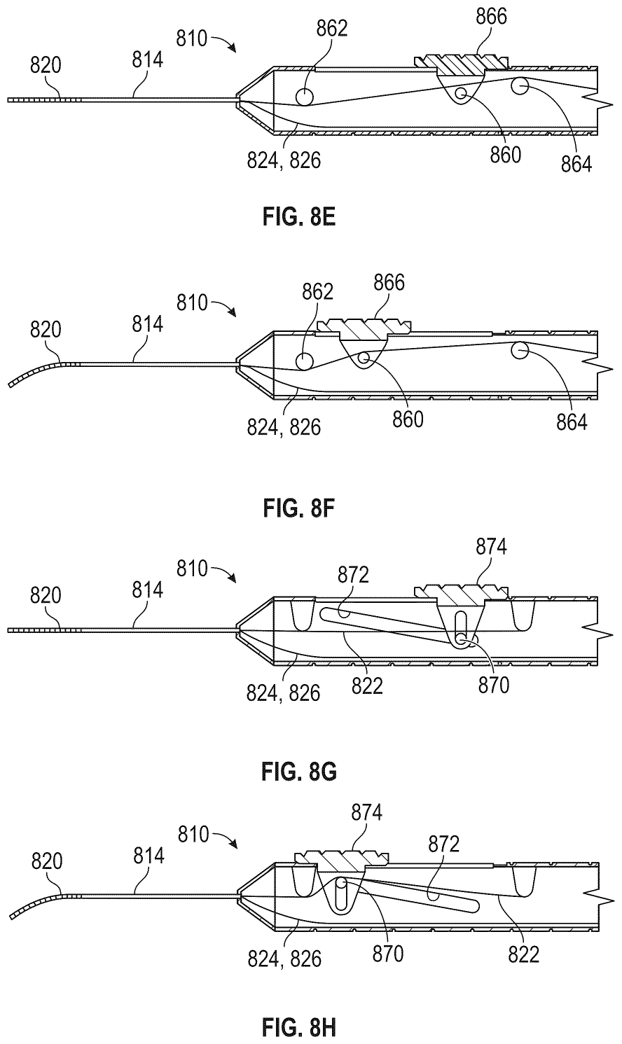

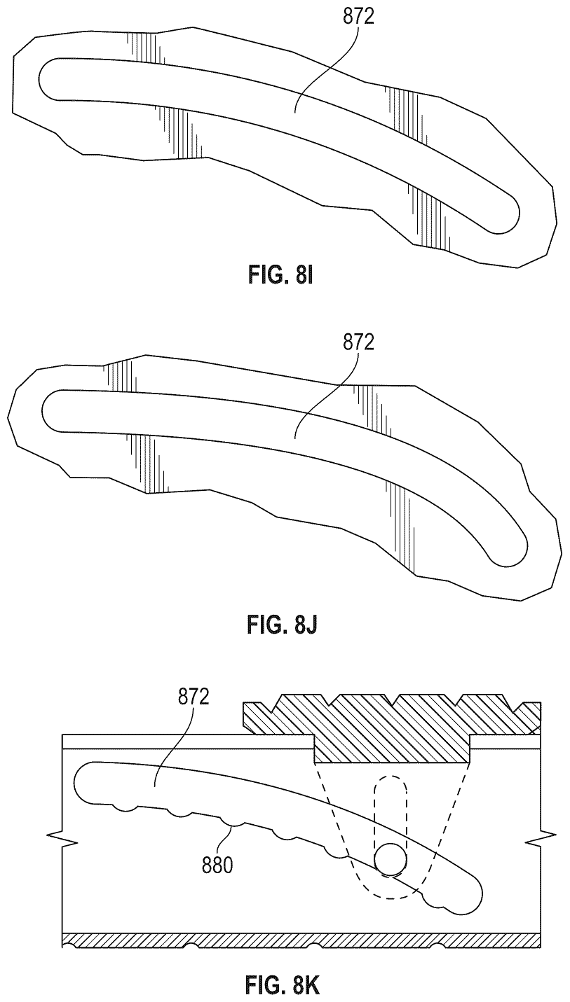

Referring now to FIGS. 8A to 8K, in accordance with certain embodiments of a surgical instrument 810 (e.g., an otologic instrument as described above) utilizing a pull-wire 822 for actuation of, e.g., articulated tube 820, various mechanisms, as will be discussed in further detail below, can be used for increasing the tension of the pull-wire 822. By way of example, as illustrated in FIGS. 8A to 8K, the surgical instrument 810 can include a canal 814 and two optical fibers 824 and 826. In some embodiments, cable 60, described above, can take advantage of the mechanisms described below with respect to the pull-wire 822.

In FIGS. 8A and 8B, the pull-wire 822 is wound on a pinion 840 secured between a control button 842 and a base 844. The pinion 840 includes two surfaces, a smaller diameter surface r which rolls between the control button 842 and a base 844, and a larger diameter surface R about which the pull wire 822 winds. The radial difference between the smaller and larger diameter surfaces r and R results in a differential displacement Δl in the pull wire as the pinion 840 rotates and translates. By selecting appropriate diameters for the smaller and larger diameter surfaces r and R, a relatively small amount of pull wire displacement Δl can be achieved during a relatively large amount of control button translation, providing the user with precise control over the deflection in an articulated tube 820, which may include a slotted tip design. In one embodiment, the smaller diameter surface r includes gear teeth with mating gear teeth on the control button 842 and the base 844. This may reduce the likelihood of slippage.

FIGS. 8C and 8D illustrate a lever arm 850 with a sliding actuation pin 852 held in place by a fixed pin 854 at a pivot of the arm. A control button can be used to advance the sliding pin 852, permitting the proximal portion of the lever arm 850 to rise, thus rotating a lanyard 856 at a distal end of the lever arm 850 to apply tension to the pull-wire 822. FIGS. 8E and 8F show a pull-wire 822 threaded over a sliding pin 860 and a first fixed pin 862 and anchored to a second fixed pin 864. Advancing a control button 866 attached to the sliding pin 860 increases the tension in the pull-wire 822.

FIGS. 8G and 8H illustrate a pull-wire 822 threaded over a sliding pin 870 that is directed in a generally upward direction by a guide track 872 as a control button 874 is advanced. The path of the guide track 872 determines how the tension in the pull-wire 822 varies as the control button is advanced, thus providing a smooth and controlled increase in tension. In the case of a linear guide, like the one illustrated in FIGS. 8G and 8H, the pull-wire take up will occur in the latter portion of the advancement of the control button 874. In the alternative configuration shown in FIG. 81, the guide track 872 is reshaped to provide greater take-up of the pull-wire at the beginning of the advancement by the control button 874 to produce a more balanced increase in tension throughout the stroke of the control button 874. In FIG. 8J, the guide track 872 inclines even more sharply so that most of the tension increase takes place early in the stroke of the control button 874. FIG. 8K illustrates an alternative embodiment of the guide track 872 with detents 880, allowing for distinct “stops” along the path corresponding to different angles of the articulated tube 820. A shelf or surface with detents can also be used with any of the embodiments of the otologic instruments presented herein using a sliding pin or similar actuation mechanism, including any of the embodiments shown in FIGS. 8A to 8K.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

Although certain embodiments refer to trans-ear canal approaches for otologic procedures, aspects described herein may also be applicable to trans-mastoid and similar approaches.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

### EXAMPLE EMBODIMENTS

Embodiment 1: A method for performing an otologic procedure comprising: inserting a cannula into a middle ear of a patient; and actuating a suction tube relative to the cannula such that a distal end of the suction tube is moved to a plurality of positions within a three-dimensional volume within the middle ear, the suction tube at least partially positioned within the cannula and at least partially extending outwardly from a distal end of the cannula.

Embodiment 2: The method of Embodiment 1, further comprising inserting the cannula at least one of: (a) through an ear canal of the patient and past a tympanomeatal flap in a transcanal procedure; or (b) through a mastoid opening in a transmastoid procedure.

Embodiment 3: The method of Embodiment 1, further comprising supplying vacuum pressure to the suction tube.

Embodiment 4: The method of Embodiment 1, further comprising rotating the suction tube relative to middle ear.

Embodiment 5: The method of Embodiment 4, wherein rotating the suction tube relative to the middle ear comprises at least one of rotating a handle to which the cannula is mounted relative to the middle ear and rotating the suction tube relative to the handle.

Embodiment 6: The method of Embodiment 1, wherein actuating the suction tube relative to the cannula comprises at least one of: (a) extending the suction tube outwardly relative to the cannula, the suction tube having a curved shape when undeformed; and (b) applying for force to at least one of a push rod and a cable extending within the suction tube and secured to the suction tube, the suction tube having one or more regions of having mechanical properties causing the suction tube to have a primary bending plane such that a first force required to bend the suction tube in a primary direction in the primary bending plane is less than 50 percent of a second force required to bend the suction tube in a plane perpendicular to the primary bending plane and intersecting a center line of the suction tube.

What is claimed is:

1. An otologic instrument comprising:
a handle configured to be held by a hand of a surgeon;
a cannula extending outwardly from the handle and mounted to the handle;
an articulated tube at least partially positioned within the cannula and at least partially extending outwardly from a distal end of the cannula; and
an actuator configured to articulate the articulated tube relative to the cannula to position a distal end of the articulated tube at a plurality of positions within a three-dimensional volume.

2. The otologic instrument of claim 1, wherein at least one of the articulated tube or the cannula is rotatably mounted to the cannula.

3. The otologic instrument of claim 2, further comprising a control structure mounted to the handle and configured to rotate the at least one of the articulated tube or the cannula in response to interaction with the control structure.

4. The otologic instrument of claim 1, wherein the cannula is mounted to a first portion of the handle, the handle including a second portion that is at least one of offset and angled with respect to the first portion.

5. The otologic instrument of claim 1, wherein:
the articulated tube has a curved shape when undeformed; and
the actuator is configured to control an amount of the articulated tube extending outwardly from the distal end of the cannula.

6. The otologic instrument of claim 5, wherein the actuator is a slider mounted to the handle.

7. The otologic instrument of claim 6, wherein the actuator is configured to move the cannula relative to the articulated tube.

8. The otologic instrument of claim 6, wherein the actuator is configured to move the articulated tube relative to the cannula.

9. The otologic instrument of claim 1, wherein the actuator is at least one of a push rod and a cable extending within the articulated tube and coupled to a control structure.

10. The otologic instrument of claim 9, wherein the articulated tube has one or more regions having mechanical properties causing the articulated tube to have a primary bending plane such that a first force required to bend the articulated tube in a primary direction in the primary bending plane is less than 50 percent of a second force required to bend the articulated tube in a plane perpendicular to the primary bending plane and intersecting a center line of the articulated tube.

11. The otologic instrument of claim 10, wherein the one or more regions include one or more slots distributed along the articulated tube.

12. The otologic instrument of claim 1, further comprising a vacuum source coupled to the articulated tube.

13. The otologic instrument of claim 1, further comprising one or more medical instruments configured to insert through the articulated tube, the one or more medical instruments selected from the group consisting of:
a tube for supplying vacuum pressure;
a tube for providing infusion fluid;
a tube for infusing at least one of medication, gel, foam, or viscoelastic material;
a tube for conducting pressurized gas;
one or more optical fibers;
a cable coupled to an ultrasonic imaging transducer; and
a waveguide for conducting electromagnetic waves for performing diathermy.

14. The otologic instrument of claim 13, wherein the one or more medical instruments comprise a plurality of medical instruments, the otologic instrument further comprising an instrument housing comprising a plurality of receptacles each configured to receive one or more of the plurality of medical instruments, the instrument housing configured to selectively guide instruments of the plurality of medical instruments into the articulated tube.
